(19) United States
(12) Patent Application Publication
(10) Pub. No.: US 2013/0000645 A1
(43) Pub. Date: Jan. 3, 2013

(54) MASK WITH PORT AND METHOD FOR USING SAME

(76) Inventor: John A. Schwind, Manasquan, NJ (US)

(21) Appl. No.: 13/536,111

(22) Filed: Jun. 28, 2012

Related U.S. Application Data
(60) Provisional application No. 61/503,333, filed on Jun. 30, 2011.
(60) Provisional application No. 61/617,418, filed on Mar. 29, 2012.

(51) Int. Cl. A62B 18/02; A61M 16/06

(52) U.S. Cl. 128/205.25; 128/206.25

(57) ABSTRACT

Embodiments of a face mask with a port configured to allow flow through the face mask at least in a direction towards a user, generally defined as an adhesive side or user side of the mask are provided, along with methods for using the same. In one embodiment, a face mask includes a breathable, flexible sheet having a first side and a second side, an adhesive disposed on a perimeter of the second side, the adhesive circumscribing a center region of the sheet and a port comprising a center region of the sheet. The port is configured to allow flow through the face mask at least in a direction towards a user without removal from the user.

### CROSS-REFERENCE TO RELATED APPLICATION

This application claims benefit of U.S. Provisional Patent Application No. 61/503,333 (Attorney Docket Number GBLS/0002L) filed on Jun. 30, 2011, and U.S. Provisional Patent Application No. 61/617,418 (Attorney Docket Number GBLS/0004L) filed on Mar. 29, 2012, which are both incorporated by reference in entirety.

### BACKGROUND OF THE INVENTION

1. Field of the Invention

Embodiments of the invention relate to a mask fabricated primarily from a breathable material and having an inlet port formed therethrough. The mask is suitable for may varied applications, such as filtered breathing, continuous positive airway pressure (CPAP) therapy, and respiratory resistance training, among others.

2. Description of the Related Art

Masks are used in a variety of applications including, but not limited to, general filtration for breathing, CPAP therapy and resistance training. Breathable masks are generally worn over the nose and/or mouth and fail to allow a user to access a user's nose and/or mouth without removing the mask. Thus, there a need for an improved breathable mask.

One example of such a need is for the treatment of sleep apnea by CPAP therapy. A CPAP device includes a pump connected by a tube to a mask that is worn over the nose and/or mouth of the wearer while sleeping. The pump provides pressure through the mask at a pressure sufficient to prevent apneas.

Most masks are worn over the nose and/or mouth and are held in place by adjustable straps. The straps must be tight enough to prevent the mask from shifting during sleep. Failure of the mask to maintain a sufficient seal may prevent sufficient pressure from being delivered to the wearer in order to prevent the apneas. Additionally, loose straps may allow leakage that dries the eyes of the wearer or may create noise which disrupts restful sleep. Thus, there is a need for an improved mask for use with CPAP therapy.

Another application includes masks used in respiratory resistance training to focus on restricted breathing. Respiratory resistance training forces a person's body to work harder to produce the required amounts of energy with less available oxygen. The body is forced to work with reduced oxygen (anaerobically). This anaerobic struggle results in physiological adaptations geared towards enhancing the efficiency of the body's respiratory, cardiovascular and oxygen utilization systems.

Until recently, little development had been made in the area of respiratory resistance training. Other than traveling to high altitudes, respiratory resistance training has been hard to perform under normal environmental conditions. Thus, there is also a need for improvement in respiratory resistance training.

### SUMMARY OF THE INVENTION

Embodiments of a face mask with a port configured to allow flow through the face mask at least in a direction towards a user, generally defined as an adhesive side or user side of the mask are provided, along with methods for using the same. In one embodiment, a face mask includes a breathable, flexible sheet having a first side and a second side, an adhesive disposed on a perimeter of the second side, the adhesive circumscribing a center region of the sheet and a port comprising a center region of the sheet. The port is configured to allow flow through the face mask at least in a direction towards a user without removal from the user.

In another embodiment, a face mask suitable for use in a CPAP system, a CPAP system and associated methods are provided. In one embodiment, a mask suitable for use with a CPAP system includes a breathable, flexible sheet having a first side and a second side, an adhesive disposed on a perimeter of the second side, the adhesive circumscribing a center region of the sheet and a hose fitting coupled to the center region of the sheet.

In another embodiment, a face mask suitable for use with a CPAP system includes a flexible sheet having a first side and a second side. An adhesive is disposed on a perimeter of the second side of the sheet, the adhesive circumscribing a center region of the sheet, the sheet having a fitting accepting aperture formed therethrough, the fitting accepting aperture having a diameter suitable to provide sufficient air for CPAP therapy.

In another embodiment, a CPAP system is provided that includes a CPAP machine, an air delivery hose and a face mask. The face mask is connectable to the CPAP machine by the hose, the mask formed from a flexible sheet having a first side and a second side, an adhesive disposed on the second side of the face mask, the adhesive circumscribing a center region of the sheet, the center region of the sheet containing a feature adapted for coupling the hose thereof. In some embodiments, the sheet may be breathable or porous.

In yet another embodiment, a method for CPAP therapy is provided that includes coupling a face mask to a CPAP machine, securing the face mask over the nose and mouth of a person, the mask secured using adhesives without the aid of straps and delivering CPAP therapy to the person through the face mask.

In still another embodiment, a method for providing a CPAP machine and providing a face mask connectable to the CPAP machine, the face mask comprising a flexible sheet having a first side and a second side, the second side of the sheet having an adhesive disposed on a perimeter, the adhesive circumscribing a center region of the sheet and an attachment feature formed in a center region of the sheet. In some embodiments, the sheet may be breathable.

A face mask suitable for use in resistance training and associated methods are also provided. In one embodiment, a face mask suitable for use in resistance training includes a breathable, flexible sheet having a first side and a second side, an adhesive disposed on a perimeter of the second side, an adhesive circumscribing a center region of the sheet and a port located in the center portion of the sheet. The port is configured to allow flow through the face mask at least in a direction towards the second side of the flexible sheet.

### BRIEF DESCRIPTION OF THE DRAWINGS

So that the manner in which the above recited features of the present invention can be understood in detail, a more particular description of the invention, briefly summarized above, can be had by reference to embodiments, some of which are illustrated in the appended drawings. It is to be noted, however, that the appended drawings illustrate only typical embodiments of this invention and are therefore not to be considered limiting of its scope, for the invention can admit to other equally effective embodiments.

FIG. 1 is a plan view of one embodiment of a face mask with port configured to allow flow through the face mask at least in a direction towards a user, generally defined as an adhesive side or user side of the mask;

FIG. 2 is a sectional view of the face mask taken along section line 2-2 of FIG. 1;

FIG. 3 is a simplified schematic of one embodiment of a CPAP system utilizing one embodiment of face mask having a port;

FIG. 4 is a plan view of the face mask of FIG. 3;

FIG. 5 is a sectional view of the face mask of FIG. 3 taken along section line 5-5 of FIG. 4;

FIG. 6 is sectional view of another embodiment of a face mask having a port;

FIG. 7 is an exploded sectional view of another embodiment of a face mask;

FIG. 8 is a side view of a person wearing one embodiment of a face mask of the present invention;

FIG. 9 is a flow diagram of one embodiment of a method for providing CPAP therapy;

FIG. 10 is another embodiment of a face mask suitable for use in a resistance training system;

FIG. 11 is a sectional view of another embodiment of a face mask suitable for use in a resistance training system;

FIG. 12 is a side view of a person wearing another embodiment of a face mask of the present invention; and

FIG. 13 is a flow diagram of one embodiment of a method for providing resistance training using a face mask of the present invention.

To facilitate understanding, identical reference numerals have been used, where possible, to designate identical elements that are common to the figures. It is contemplated that elements and features of one embodiment may be beneficially incorporated in other embodiments without further recitation.

It is to be noted, however, that the appended drawings illustrate only exemplary embodiments of this invention and are therefore not to be considered limiting of its scope, for the invention may admit to other equally effective embodiments.

### DETAILED DESCRIPTION

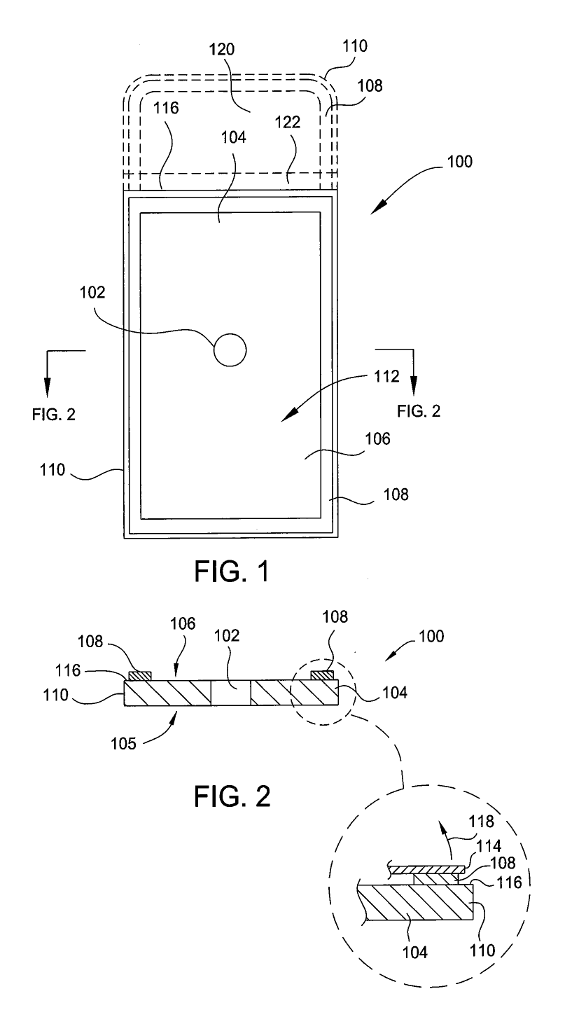

FIG. 1 depicts a rear elevation of one embodiment of a face mask 100 with a port 102. The port 102 is generally configured to allow flow through the face mask at least in a direction towards a user, generally defined as an adhesive side or user side of the face mask. The face mask 100 is generally fabricated from primarily a flexible sheet 104 having a sufficient surface area to cover the mouth and nose of a user. The sheet 104 in its unused state may have a performed contour, or be flat and planer. The sheet 104 is strong and has sufficient flexibility to conform to the face of the user, thereby increasing comfort and minimizing air leaks. The sheet 104 may be fabricated from one or more layers of a melt blown material, spun bond material, wet laid glass material, cellulous material, carbon cloth or other suitable filtration media or air permeable material. It is also contemplated that the sheet 104 may comprise a plurality of breathable layers laminated together. The laminated layers comprising the sheet 104 may be quilted, spot welded or affixed in another manner which substantially prevents delamination. Examples of sheets 104 which may be adapted to benefit from the invention are described in U.S. Pat. No. 6,609,516, issued Aug. 26, 2003, which is incorporated by reference in its entirety. In one embodiment, the flexible sheet 104 may be breathable, such as being porous or a comprised of filtration media. The use of porous media for the sheet 104 allows for the continuous exchange of fresh air, proving comfort to the user while filtering out air on exhalation thereby reducing the spread of germs. In one embodiment, the sheet 104 has a filtration efficiency of at least 95% of 0.3 micron particles at 85 liters per minute flow rate. In another embodiment, the sheet 104 may have a MERV 4 rating or higher. In certain embodiments, the sheet 104 may not be porous.

As noted above, the port 102 is configured to allow flow through the face mask at least in a direction towards a user, generally defined as an adhesive side or user side of the face mask. The port 102 may be configured to couple with a conduit, such as a hose or tube, or be configured to be opened and closed. In one embodiment, the port 102 is configured to allow the entry of a fluid, for example, liquid or gas, to pass through the face mask 100 facing minimal resistance from the sheet 104 to a user and allow preferential passage of the fluid relative to the surrounding material of the sheet 104. The port 102 may be a circular cut-out, slit or other hole through the sheet 104. In some embodiments, the port 102 may be configured to be sealable, for example, by a valve or sealing member, which may be opened to allow flow through the face mask at least in a direction towards a user.

Referring to FIGS. 1 and 2, the flexible sheet 104 primarily comprising the face mask 100 includes a front side 105 and a back side 106. The back side 106 generally is intended to face the user when the face mask 100 is worn. The flexible sheet 104 may have a rectangular plan form and be flat when not in use. Alternatively, the sheet 104 may have other plan forms. In one embodiment, a ring-shaped adhesive layer 108 is disposed on the back side 106 of the sheet 104 proximate an edge 110 of the sheet 104, circumscribing a center region 112 of the sheet 104. The adhesive layer 108 is utilized to secure the sheet 104 to the face of the user to achieve a uniform seal around the perimeter of face mask 100. Suitable adhesives include pressure sensitive adhesives of the type suitable for adhesion to human skin, and releasable from human skin without injury, such as medical grade temporary adhesives. In one embodiment, the adhesive is a silicon adhesive which allows the face mask 100 to be reusable. The silicone adhesive advantageously allows the face mask 100 to be washed, stored in a manner where the mask may be folded to allow adhesive to adhesive contact, and reused. In another embodiment the adhesive is hydrogel adhesive suitable for adhesion to human skin and allows the user to sweat or come in contact with liquids and retain the effectiveness of the hydrogel adhesion. Any of the adhesives described above may be used on any of the masks described herein. However, other types of adhesives may be suitable as well.

In embodiments wherein the sheet 104 is breathable, the edge 110 may comprise a ring (not shown) of material circumscribing the center breathable region 112 to provide a surface of deposition of the adhesive layer 108.

A release liner 114 (only shown in the enlarged portion of FIG. 2) may be disposed over the ring-shaped adhesive layer 108 when the face mask 100 is not in use. The release liner 114 may be removed from the sheet 104 to expose the adhesive layer 108 for securing the face mask 100 to the user.

The sheet 104 may include an adhesive-free narrow border 116 defined between the edge 110 and adhesive layer 108. The adhesive-free border 116 provides a gripping surface to assist peeling of the release liner 114 from the adhesive layer 108, as indicated by arrow 118, and to assist removal of the face mask 100 from the user. The release liner 114 and the adhesive-free border may be incorporated in any of the embodiments of masks described herein.

In one embodiment the face mask 100 optionally includes a transparent eye guard 120, shown in phantom in FIG. 1. The guard 106 may be made of transparent plastic or any other suitable transparent material for guarding a user's eyes from external factors, such as smoke or other eye irritant. In one embodiment, the guard 106 may be made of an anti-fog material. The adhesive layer 108 is utilized to secure the guard 120 to the face of the user to achieve a uniform seal around the perimeter of face mask 100. The adhesive layer 108 and the edge 110 extend upwards from the flexible sheet 104 around the perimeter of the guard 120. The guard 120 may be affixed to the mask by any suitable attachment means, for example, such as adhesives or sonic welding. The guard 120 may be incorporated in any of the embodiments of masks described herein.

In another embodiment, the face mask 100 optionally includes a stiffening member 122, shown in phantom FIG. 1, coupled to the front side 105 to assist in removal of the face mask 100 from the user. In one embodiment, the stiffening member 122 may be affixed to the top edge 110 of the sheet 104. The stiffening member 122 may be a printable material and/or be a reflective material to enhance safety of the user if using along roadways. The stiffening member 122 may be incorporated in any of the embodiments of masks described herein.

The face mask 100 may be utilized or adapted for utilization in resistance training, CPAP therapy, personal protection, medial use, security and police use, among others. The face mask 100 allows for fast and easy deployment, while being extremely portable and easily stowed or carried.

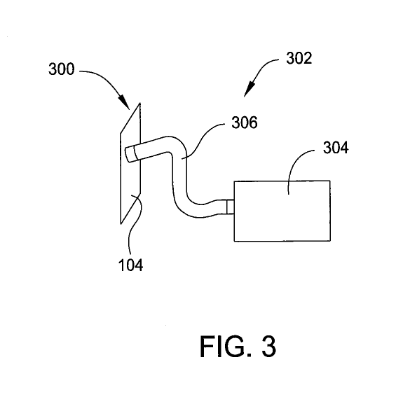

FIG. 3 depicts one embodiment of a face mask 300 having port 102 is configured to accept a conduit, for example, a tube or a hose. The face mask 300 may be coupled by the conduit to a gas or liquid source, such as an oxygen supply, anesthetic supply or other gas supply. In the embodiment depicted in FIG. 3, the face mask 300 is schematically illustrated as part of a CPAP system 302. The CPAP system 302 includes a CPAP machine 304 connected by a hose 306 to the face mask 300, which may be strapless due to the use of the adhesive layer 108. The CPAP machine 304 is operable to provide sufficient air pressure to a person wearing the face mask 300 sufficient to provide CPAP therapy to the person. It is contemplated that the CPAP machine 304 may be any commercially available CPAP machine or other suitable CPAP air source. Suitable CPAP machines are available from Phillips Respironics, among others. The face mask 300 is detachably connected to the hose 306. In one embodiment, the face mask 300, which may be permeable or impermeable to air, is configured to be affixed to the face of a person being treated for sleep apnea without the use of straps. Moreover, the lack of straps increases the comfort level of the wearer while minimizing the potential shifting and leakage of the face mask 300 due to movement of the strap.

The face mask 300 includes a sheet 104 constructed as described above. In one embodiment the sheet 104 may be a non-permeable flexible web of material. In another embodiment, the flexible sheet 104 is permeable and has sufficient resistance to air flow in order to provide the wearer with a continuous flow of air at a pressure sufficient to prevent apnea from occurring.

In one embodiment, the sheet 104 is permeable and muffles air passage during a users' exhalation, thereby providing a more restful sleep as compared to conventional masks. Additionally, the use of engineered breathable media for the sheet 104 allows for the correct pressure to be maintained within the face mask 300 for optimal breathing and treatment.

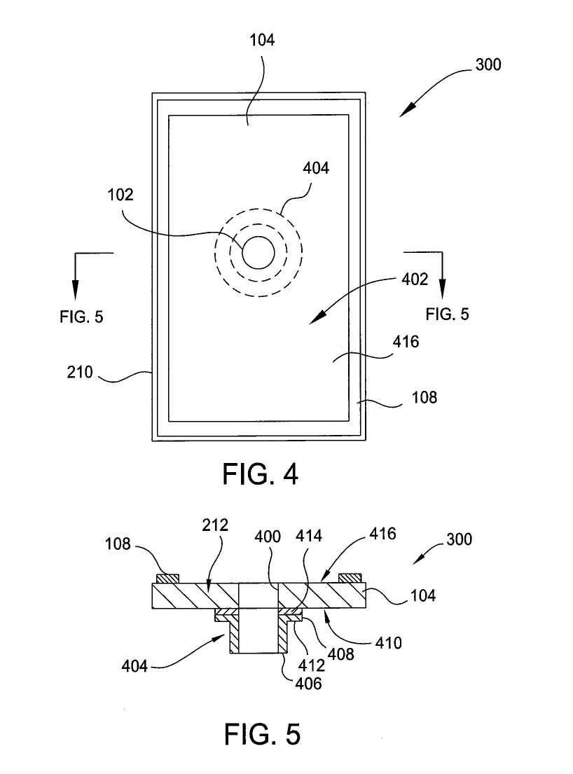

FIG. 4 is a rear elevation of the face mask 300, and FIG. 5 is a cross-sectional view of the face mask 300 taken through section line 5-5 of FIG. 4. Referring to FIGS. 4 and 5, in one embodiment, the port 102 is a hose fitting attachment feature 400 formed in a center region 402 of the sheet 104. The hose fitting attachment feature 400 may not necessarily be in the geometric center of the center region 402.

A fitting 404 is generally interfaced with the fitting attachment feature 400 of the sheet 104. The fitting 404 has a first end 406 which is configured to permit coupling of the fitting 404 to the hose 306. A second end 408 of the fitting 404 is configured to facilitate attachment of the fitting 404 to the sheet 104 such that the second end 408 of the fitting 404 is disposed on a front side 410 of the sheet 104. The first end 406 of the fitting 404 may be configured to mate with the hose 306 in a conventional manner, such as a press fit, thread, bayonet fitting, quarter turn or other suitable hose connection.

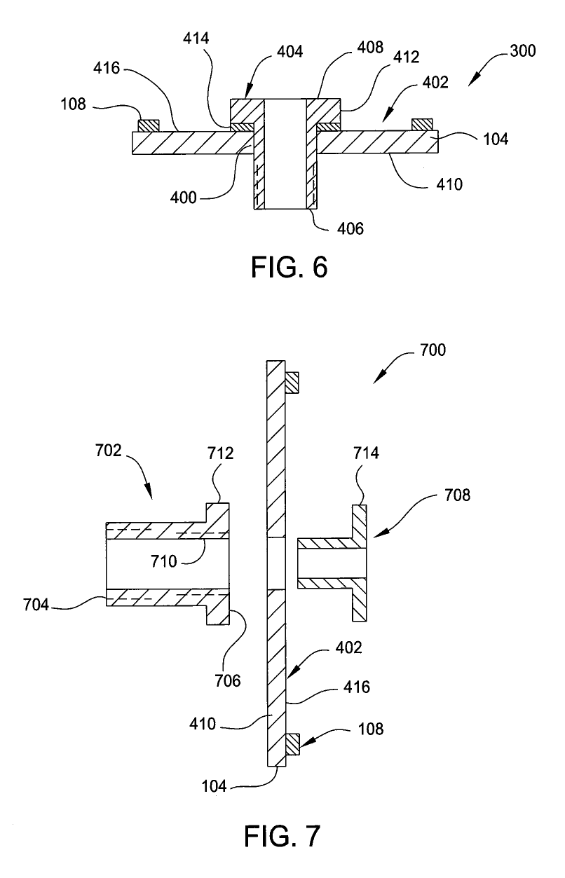

The second end 408 of the fitting 404 includes a flange 412. The flange 412 facilitates affixing the fitting 404 to the flexible sheet 104. In one embodiment, an adhesive 414 may be disposed between the flange 412 and the sheet 104 and the front side 410 of the sheet 104 as shown in FIG. 5. Alternatively, the flange 412 may be affixed to the front side 410 of the sheet 104 by sonic welding, stitching, clamping or other suitable method. It is also contemplated that the flange 412 may be coupled to a back side 416 of the sheet 104 via one of the methods described above as shown in FIG. 6.

Continuing to refer to FIG. 5, the fitting 404 is generally aligned with the feature 400 to facilitate the flow of air from the hose 306 through the fitting 404 to the user on the back side of the face mask 300. The fitting 404 may be configured as a universal fitting or to include adapters such as to allow for different types of hose connections and use with different types of CPAP machines 304. In one embodiment, the fitting 404 mates with hose 306 (or second fitting coupled to the end of the hole 306) via a thread, quarter turn connection, bayonet connection, swag fitting or other suitable mating arrangement.

In the embodiment depicted in FIGS. 4-6, the fitting 404, once attached to the sheet 104, becomes an integral portion of the face mask 300. Thus, a user may readily disconnect the fitting 404 of the face mask 300 from the hose 306 and attach a new mask to the hose 306 and CPAP machine 304 once the service life of the first mask has expired. Since the sheet 104 of the unused face mask 300 in one embodiment is flexible and flat, the face mask 300 may be packed flat or folded in flat poly bags or sleeves, thereby facilitating high density packing and convenient storage.

FIG. 7 is an exploded sectional view of another embodiment of a mask 700 which may be utilized with the CPAP system 302 described above. The mask 700 includes a flexible sheet 104 constructed as described above. The mask 700 is substantially similar to the face mask 300 except that the fitting 702 is not permanently affixed to the sheet 104. In this manner, only the sheet 104 of the mask 700 needs to be replaced at the end of the service life, wherein the fitting 702 removed from the old mask may be reused with replacement mask.

In the embodiment depicted in FIG. 7, the fitting 702 includes a first end 704 detachably couple-able to the hose 306 and a second end 706 configured to detachably affix to the first end of the fitting 702 in a manner which captures the sheet 104 to the fitting 702. In the embodiment illustrated in FIG. 7, the second end 706 of the fitting 702 includes a male threaded tab 708 which engages a female threaded portion 710 formed in the second end 706. When the tab 708 is threaded into the threaded portion 710, flanges 712, 714 of the tab 708 second end 706 are displaced toward each other, thereby capturing the sheet 104 therebetween. When a user wishes to replace the mask 700, the tab 708 may be removed from the second end 706, thereby releasing the sheet 104 and allowing the fitting 702 to be reassembled, capturing a new sheet therebetween.

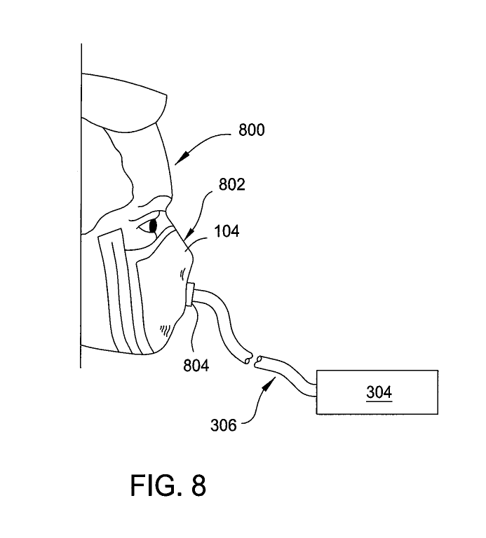

FIG. 8 depicts a side view of a user 800 wearing a face mask 802 of the present invention. The face mask 802 may be any of the masks described herein. The adhesive layer 108 on the back side of the face mask 802 allows the sheet 104 to flexibly conform to the user's face, thereby providing a substantially air-tight seal around the mouth and nose of the user. Since the face mask 802 does not include and/or need straps to hold the face mask 802 in place, the user's comfort level is increased while the probability of leakage due to straps falling at the mask is eliminated. A fitting 804 extending from the front side of the face mask 802 allows the hose 306 to be readily connected to the mask either before or after the hose is connected to the CPAP machine 304. The fitting 804 may be configured as described above. Thus, CPAP therapy may be provided by the system 302 with greater comfort to the user and with less probability of leakage.

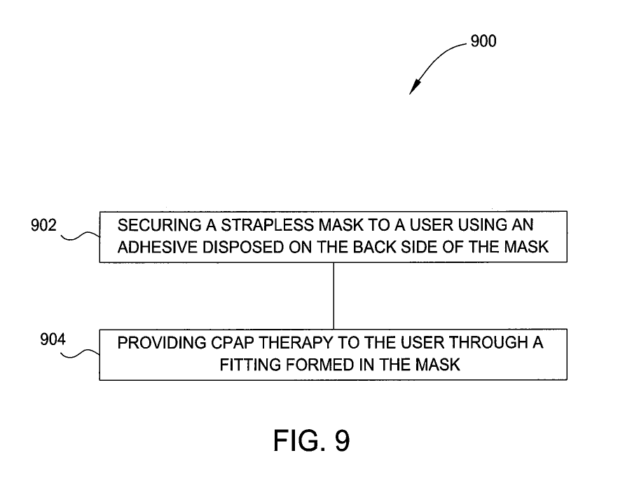

FIG. 9 depicts a flow diagram of one embodiment of a method for providing CPAP therapy 900 using CPAP machine 304, hose 306 and strapless face mask 802. The method begins at step 902 by securing the strapless face mask 802 to a user 800 using an adhesive disposed on the back side of the face mask 802. At step 904, a CPAP therapy is provided by providing air from the CPAP machine 304 to the user 800 through a fitting formed in a center region of the face mask 802 which is surrounded by the adhesive.

The strapless face mask provides increased user comfort while minimizing potential leakage. Furthermore, the mask's flat form allows for easy packaging and storage. Additional benefits of the strapless mask include a substantially leak free seal which prevents pressure lost which causes conventional CPAP masks to stops working; comfortable fit without leakage and over tightening reduces skin irritation and pressure sores, improved hygiene due to single use and elimination of daily cleaning required by conventional CPAP masks. The strapless mask also allows a more natural sleeping position. Sleeping on your back is the worst position for sleep apnea. Sleeping on your side can put pressure on existing hard CPAP masks, which can break the seal and causing the mask to leak air. The flexibility of the strapless mask allows the user to sleep comfortably on their side. The strapless mask being substantially flat is easy to ship and store, with one size fits all—thereby eliminating need for custom, expensive fittings. The same strapless mask will accommodate both mouth and non-mouth breathers. The strapless mask is easy to get used to and reduces and/or eliminates claustrophobic feelings sometimes associated with conventional CPAP masks.

As discussed above, other embodiments of the mask include a port that is configured to be sealable, for example, by a valve or sealing member, so that the port may be selectively open and closed. The port, when in the open or unsealed state, is configured to allow flow through the face mask at least in a direction towards a user, generally defined as an adhesive side or user side of the face mask.

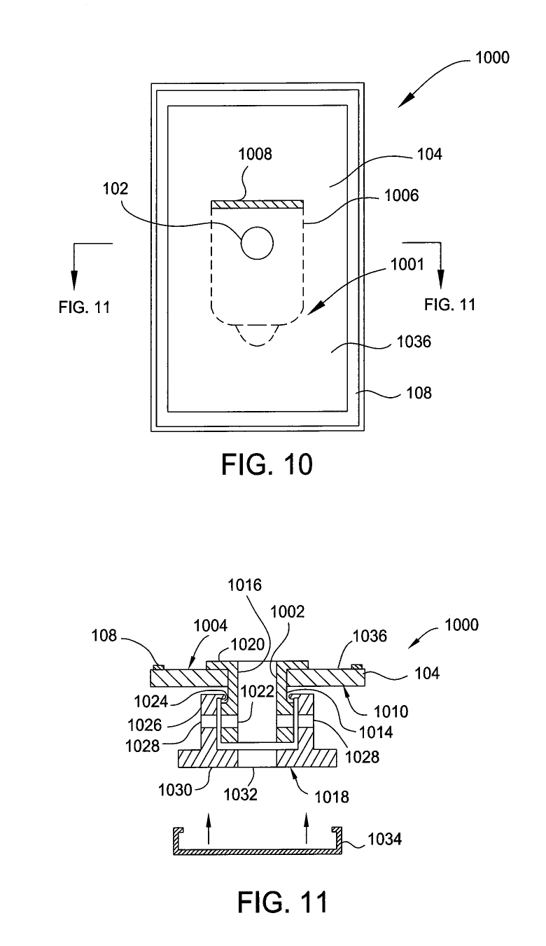

FIG. 10 is a rear elevation of another embodiment of a face mask 1000. The face mask 1000 is suitable for use in a resistance training system. The face mask 1000 is primarily fabricated from a flexible sheet 104 that is air permeable, and is configured to be affixed to the face of a person performing resistance training without the use of straps. The lack of straps increases the comfort level of the wearer while minimizing the potential shifting and leakage of the face mask 1000 during resistance training. In one embodiment, the face mask 1000 includes the flexible sheet 104 constructed as described above, and may be breathable, such as being porous. In one embodiment, the resistance level of the sheet 104 for inhalation has a range of between about 3.5 mm H₂O to about 10.5 mm H₂O based upon the National Institute for Occupational Safety and Health (NIOSH) test procedure RCT-APR-STP-0007, now TEB-APR-STP-0007.

The face mask 1000 may be provided as part of a kit comprising two or more different types of masks 1000, each type of face mask 1000 having sheet 104 having a different flow resistance to allow the user to select an appropriate resistance mask for a particular training need and/or to include masks of varying resistance, such as low, medium and high resistance sheets, to facilitate training regimes which require different resistances at different training stages. In one embodiment, the mask types comprising the kit may be selected from mask types having a flow resistance based upon the NIOSH test procedure TEB-APR-STP-0007 in a range of between about 3.5 mm H₂O to about 5.5 mm H₂O for a first mask type, a range of between about 5.5 mm H₂O to about 6.5 mm H₂O for a second mask type, a range of between about 6.5 mm H₂O to about 7.5 mm H₂O for a third mask type, and a range of between about 7.5 mm H₂O to about 10.5 mm H₂O for a fourth mask type.

The sheet 104 muffles air passage during the users' exhalation, thereby providing resistance even during exhalation. Optionally, a flapper-type check valve (or other suitable valve, not shown) may be affixed to the sheet 104 to allow substantially unrestricted exhalation.

Referring to FIGS. 10 and 11, the port 102 is a sealable port 1002 formed in the center region 1004 of the sheet 104 in face mask 1000. The port 1002, in its open state, is configured to allow the flow of fluids through the mask to the user. In one embodiment the centerline of the port 1002 is located within around one inch of the centerline of the face mask 1000 so that it is aligned to where a user's mouth is located to facilitate use of the port 1002 to deliver water or other thirst quenching liquid to the user without removal of the face mask 1000 or partial peeling of the mask from the face of the user. In the embodiment depicted in FIG. 11, the port 1002 is in the form of an aperture formed through the center region 1004 of the sheet 104. As described above, the port 1002 may be a circular cut-out, slit or other hole through the sheet 104 which allows preferential passage of air relative to the surrounding material of the sheet 104 comprising the center region 1004. The port 1002 may be an elastomeric member that is bonded to the flexible sheet 104 which may be sealingly opened and closed.

In one embodiment, the port 1002 may be covered by a seal 1006 on a front side 1010 of the face mask 1000 (shown in phantom in FIG. 10). In one embodiment, the seal 1006 is in the form of a flap flexible material that operates to open and close access to the port 1002. In one embodiment, the seal 1006 is securely attached to the flexible sheet 104 by an attachment 1008, which may be a sonic weld or an adhesive strip. The seal 1006 has a temporary adhesive layer that removably secures the flap (e.g., seal 1006) to the sheet 104, such that the seal 1006 covers and substantially seals the port 1002. The seal 1006 may also include an adhesive free tab 1012 positioned on the opposite side of the port 1002 from the attachment 1008 to assist in opening and closing the seal 1006. The temporary adhesive layer may be of the same material as described for the adhesive layer 108. The seal 1006 may be incorporated in any of the embodiments of masks described herein.

In another embodiment, the seal 1006 may be in the form of a valve 1014 interfaced with the port 1002 of the sheet 104. The valve 1014 includes a valve body 1016 and a control knob 1018. The valve body 1016 includes a flange 1020 and one or more valve apertures 1022 formed therethrough. The flange 1020 is sealingly coupled to the sheet 104. The flange 1020 may be affixed to the sheet 104 by sonic welding, stitching, crimping or other suitable method. In one embodiment, an adhesive may secure the flange 1020 and the sheet 104. The valve 1014 has an undercut 1024 which is configured to retain the knob 1018 while allowing the knob 1018 to be turned to control the flow of air through the valve 1014, and hence, through the port 1002 of the face mask 1000.

In one embodiment, the knob 1018 includes a knob flange 1026, one or more knob apertures 1028, and a knob base 1030. The knob flange 1026 engages the undercut 1024 to retain the knob 1018 to the valve 1014. The one or more knob apertures 1028 are located between the knob base 1030 and knob flange 1026. The knob base 1030 includes a knob opening 1032 to allow liquids to be delivered to a user's mouth through the valve 1014 and port 1002. As seen in FIG. 11, the knob opening 1032 may be selectively sealed by a cap 1034 at the knob base 1030. In another embodiment, the cap 1034 is coupled to the knob 1018 by a living hinge (not shown). The cap 1034 may be incorporated in any of the embodiments of masks described herein.

In operation, the valve 1014 has at least three operational states. In one operational state, the knob 1018 may be turned to control the amount of alignment between the valve aperture 1022 of the body 1016 and the apertures 1028 of the knob 1018, thus allowing the amount of air flowing through the valve 1014 and port 1002 to the user to be controlled, thereby controlling the ultimate flow resistance through the face mask 1000 (as shown in FIG. 11). When the knob 1018 is turned to prevent the valve apertures 1022 from aligning with the knob apertures 1028, flow is substantially prevented through the valve 1014 and along the flow of air through the sheet 104 of the face mask 100.

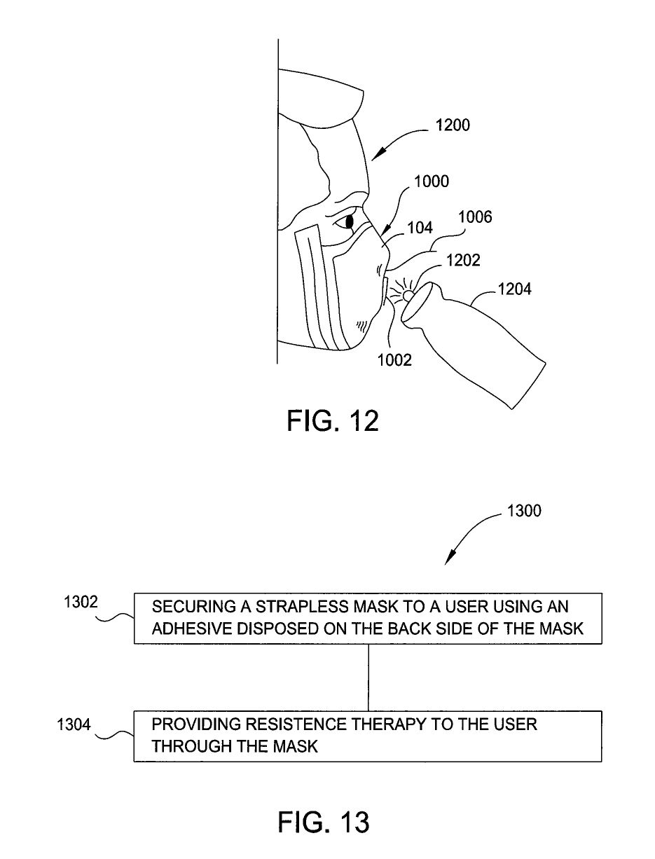

FIG. 12 depicts a side view of a user 1200 wearing the face mask 1000 of the above embodiment, however the mask may be any of the masks described herein. The adhesive layer 108 on a back side 1036 (shown in FIGS. 10 and 11) of the face mask 1000 allows the sheet 104 to flexibly conform to the user's face, thereby providing a substantially air-tight seal around the mouth and nose of the user. Since the face mask 1000 does not include and/or need straps to hold the face mask 1000 in place, the user's comfort level is increased while the probability of leakage due to straps falling at the mask is eliminated. In one embodiment, the seal 1006 may be opened to allow a nipple 1202 of a water bottle 1204 or a straw to deliver liquid into the mouth of the user 1200.

FIG. 13 depicts a flow diagram of one embodiment of a method 1300 for providing resistance therapy using the strapless face mask 1000. The method 1300 begins at step 1302 by securing the strapless mask to the user 1200 using an adhesive disposed on the back side of the mask. At step 1304, resistive therapy is provided by wearing the mask during training activities such as bike exercise, running, jogging, sparring and the like.

Reducing the oxygen available using the different operational states duplicates the effects of training at high altitude. The big challenges with high altitude training are the expense, commitment and the exposure for someone who has not yet acclimatized. The mask provides benefits similar to high altitude training for resistive training in a more cost effective and safe manner.

While the foregoing is directed to embodiments of the present invention, other and further embodiments of the invention can be devised without departing from the basic scope thereof, and the scope thereof is determined by the claims that follow.

What is claimed is:

1. A face mask comprising:
a flexible sheet having a first side and a second side, the flexible sheet being permeable to air;
an adhesion layer disposed on a perimeter of the second side, the adhesion layer circumscribing a center region of the sheet; and
a port formed through the center region of the sheet, the port configured to allow fluid to flow through the sheet in a direction from the first side to the second side.

2. The face mask of claim 1, further comprising:
a fitting coupled to the port, the fitting configured to accept a hose or tube.

3. The face mask of claim 1, further comprising:
a sealing member adapted to selectively seal the port.

4. The face mask of claim 1, further comprising:
a transparent eye guard.

5. The face mask of claim 1, wherein the adhesion layer further comprises:
a pressure sensitive adhesives of a type suitable for releasable and reusable adhesion to human skin, and releasable from human skin without injury.

6. The face mask of claim 1, wherein the adhesion layer further comprises:
a medical grade temporary silicon adhesive.

7. The face mask of claim 1, wherein the adhesion layer further comprises:
a hydrogel adhesive suitable for adhesion to human skin.

8. A CPAP system, comprising:
a face mask comprising:
a flexible sheet having a first side and a second side;
an adhesive disposed on a perimeter of a second side of the flexible sheet, the adhesive circumscribing a center region of the flexible sheet; and
a fitting engagement feature formed through the central portion of the sheet;
a CPAP machine; and
a hose adapted for coupling the face mask to the CPAP machine.

9. A method for providing CPAP therapy, comprising:
securing a strapless face mask to a user; and
proving CPAP therapy to the user through the strapless face mask.

10. A face mask suitable for resistive training, the mask comprising:
a flexible sheet having a first side and a second side, the flexible sheet being permeable to air;
an adhesion layer disposed on a perimeter of a second side of the flexible sheet, the adhesion layer circumscribing a center portion of the flexible sheet; and a
a port formed through the central portion of the sheet, the port configured to allow fluid to flow through the sheet in a direction from the first side to the second side.

11. The face mask of claim 10, further comprising:
a sealing member operable to open and close access to the port.

12. The face mask of claim 10, further comprising:
a stiffening member coupled to the flexible sheet.

13. The face mask of claim 10, wherein the sheet further comprises an adhesive-free border defined between an edge of the mask and the adhesion layer.

14. The face mask of claim 10, wherein the flexible sheet comprises filter media.

15. The face mask of claim 10, further comprising a valve interfaced with the port, wherein the valve is operable to selectively control the amount of air flowing into and out of the port.

16. The face mask of claim 15, further comprising a sealing member operable selectively close the port.

17. The face mask of claim 10, wherein the adhesion layer further comprises:
a pressure sensitive adhesives of a type suitable for releasable and reusable adhesion to human skin, and releasable from human skin without injury or discomfort.

18. The face mask of claim 10, wherein the adhesion layer further comprises:
a medical grade temporary silicon adhesive.

19. The face mask of claim 10, wherein the adhesion layer further comprises:
a hydrogel adhesive suitable for adhesion to human skin.

20. A kit comprising a plurality of face masks having at least two different flow resistances through sheets comprising the face masks, wherein one of the plurality of face masks is the face mask of claim 10.
